(19) United States
(12) Patent Application Publication
(10) Pub. No.: US 2007/0027403 A1
(43) Pub. Date: Feb. 1, 2007

(54) A Method and System of Continual Temperature Monitoring

(75) Inventor: Dale Herman Kosted, Enid, OK (US)

Correspondence Address: Dale Kosted, D.V.M., 3502 King St., Enid, OK, 73703, US

(73) Assignee: Dale Herman Kosted, Enid, OK (US)

(21) Appl. No.: 11/161,292

(22) Filed: Jul. 28, 2005

(51) Int. Cl. A61B 5/00

(52) U.S. Cl. 600/549

(57) ABSTRACT

A method and system of Continual Temperature Monitoring of a patient pre-surgical, intra-surgical, and post-surgical by multiple, wireless, temperature sensors that transmit low-power radio frequencies to a monitor capable of receiving signals of temperature data, wherein the monitor interfaces with conventional PC platforms having specifically designed software, and the multiple wireless sensors are of two types: 1.) a capsule shaped sensor which can be ingested or inserted rectally which transmits core body temperature (CBT); 2.) a dermal patch which transmits external body temperature, and the PC software allows the user to set upper and lower limits for each sensor transmitter placed in or on a patient which will activate a notification method selected by the user if these limits are exceeded or subceeded, and the PC software has file transfer protocol (FTP) allowing the user with an internet connection from any location worldwide to access a secure, password protected intranet or internet website and monitor, in real-time, the temperature data of a patient.

### FIELD OF THE INVENTION

The present invention relates generally to uses of temperature monitoring devices, and more particularly to a method and system for continual temperature monitoring of a patient pre-surgical, intra-surgical, and post-surgical, by multiple, wireless, temperature sensors that transmit low-power radio frequencies to a monitor which interfaces with conventional PC platforms having specifically designed software allowing the user: to set limitations; to set methods of notification; to view temperature data on a secure, password protected website in real-time via an intranet or internet connection.

### BACKGROUND OF THE INVENTION

Many temperature devices have been designed with various functions to aid In monitoring a patient, and they have been widely described in the Prior Art. See for example U.S. Pat. Nos. 6,629,776; 5,050,612; 4,819,860; 4,387,724; 4,297,685; 4,270,547; 4,151,831; United States Patent Application 20020107557. The above patents, in some cases, disclose systems for a continual temperature monitoring of a patient. However, these temperature monitors only monitor either an internal, core body temperature, or an external body temperature.

The pulmonary artery has been recognized as an ideal site for monitoring core body temperature because in the heart the blood is mixed from the viscera and the skin. However, inserting a Swan-Ganz catheter to monitor temperature in the pulmonary artery is also the most invasive procedure there is for the patient. The bladder is also recognized as an ideal site for monitoring core body temperature. However, inserting a urinary bladder sensor catheter is too invasive for just monitoring CBT. Other core accessible sites are the esophagus and the tympanic membrane. None of these sites are convenient for continual temperature monitoring or comfortable for a patient.

The placing of a sensor in the esophagus or intra-tracheal is considered invasive and is commonly used during surgery or in critical care areas. However, due to the length of the esophagus the placement of the sensor is very important. If it is placed too high in the esophagus the reading will be affected by tracheal air or just the effects of anesthetic gases can alter true CBT.

Tympanic membrane sensors, even though considered non-invasive, are inaccurate and insensitive in identifying patients with a “rectal-based” fever. Also, it has been recognized that the size of the ear canal, the technique used in directing the infra-red reader, and metabolic occurrences, such as the presence of an infection, can affect the accuracy of the temperature reading. Also, otic infections can also give elevated temperature readings while CBT is normal. Therefore, a rectal sensor is conclusively the most convenient site for continual temperature monitoring.

All of the above patents fail to identify the need to monitor during surgery extremity temperature and tie it together with core body temperature (CBT) in an integrated temperature measuring system. The core body temperature(CBT) of a patient is critical before, during, and after surgery. Also, since external heating devices are used to warm patients, it is important that adequate monitoring extremities of a patient is done to make sure the extremities are kept warm because they are the first area sacrificed by the mammalian body in order to maintain CBT. Also, there are numerous incidences recorded where these external heating devices such as warming blankets, heating pads, etc., have malfunctioned resulting in severe burns of skin in contact with them where if a monitoring system had been in place giving early notification that extremity body temperature was entering the dangerous range these burns could have been avoided.

### SUMMARY OF THE INVENTION

An object of the present invention is to provide a method and system for continual temperature monitoring of a patient pre-surgical, intra-surgical, and post-surgical via multiple, wireless, temperature sensors that transmit low-power radio frequencies to a monitor capable of receiving signals of temperature data which interfaces with conventional PC platforms having a specifically designed software. The multiple, wireless, temperature sensors are of two types: 1.) a capsule shaped sensor which can be ingested or inserted rectally which transmits core body temperature (CBT); 2.) a dermal patch which transmits external body temperature. The PC software allows the user to set upper and lower limits for each sensor transmitter placed in or on a patient which will activate a notification method selected by the user if these limits are exceeded or subceeded. Furthermore, the PC software has file transfer protocol (FTP) allowing the user with an internet connection from any location worldwide to access a secure, password protected intranet or internet website and monitor, in real-time, the temperature data of a patient.

Advances in existing technology have resulted in temperature sensors the size of a capsule that can be inserted rectally for CBT measurements and that transmit said temperatures wirelessly via telemetry to a receiver. Dermal patches exist that transmit external body temperature wirelessly via telemetry to a receiver. The use of combining these two existing technologies for an integrated method and system for continual temperature monitoring of a patient is novel, and those skilled in the prior art have been unaware of such an application. Interfacing this technology with a PC which has software designed to publish in real-time to an intranet or internet website so that the user can view from any location worldwide also is a novel application of this technology. And, the present invention provides a novel method and system of monitoring CBT and external body temperature in the operating room during surgery. And, the present invention provides the user a means of selecting the best method of notification when the patient has exceeded or subceeded limitations considered critical to accurately and continuously monitor for hypothermia/hyperthermia with the goal of striving for normothermia on selected patients. Such methods of notification are, not excluding any future alarm systems developed, a telephone call, a cell phone, an e-mail, a pager, an alarm that can sound during surgery in a operating room if the limits are exceeded or subceeded.

### DETAILED DESCRIPTION OF THE INVENTION

The method of the present invention is as follows:
1.) A capsule shaped sensor transmitter is initialized and activated then ingested or inserted rectally in a pre-surgical patient; and,
2.) a dermal patch sensor transmitter is initialized and activated then placed on the skin of a patient in various locations, primarily the extremities.
   The System Comprises:
1.) a capsule shaped sensor that transmits core body temperature data which can be ingested or inserted rectally which transmits low-power radio frequencies to a monitor;
2.) a dermal patch sensor which transmits external body temperature data by low-power radio frequencies to a monitor;
3.) a monitor which: initializes and activates the sensors; receives the low-power radio frequencies of temperature data from the capsule shaped sensor and the dermal patch sensors; capable of interfacing with conventional PC platforms to transfer received temperature data;
4.) software specifically designed which: downloads temperature data from the monitor; allows user to set temperature limitations with a method of notification when these limits are exceeded or subceeded; has file transfer protocol that publishes in real-time to a secure, password protected intranet or internet website for user viewing; is compatible with conventional PC platforms.

What is claimed is:

1. A method and system of continual temperature monitoring of a patient pre-surgical, intra-surgical, and post-surgical by multiple, wireless, temperature sensors that transmit low-power radio frequencies to a monitor, capable of receiving signals of temperature data, which interfaces with conventional PC platforms and has specifically designed software.

2. said multiple, wireless, temperature sensors of claim 1 are of two types:
1.) a capsule shaped sensor;
2.) a dermal patch sensor.

3. the capsule shaped sensor of claim 2 can be ingested or inserted rectally.

4. the dermal patch sensor of claim 2 is placed on the skin of the patient.

5. the capsule shaped sensor of claim 2 records and transmits core body temperature data to said monitor of claim 1.

6. the dermal patch sensor of claim 2 record and transmits external body temperature data to the monitor of claim 1.

7. the monitor of claim 1 activates and initializes the temperature sensors.

8. said software of claim 1 allows the user to set upper and lower limits for each temperature sensor which will activate notification methods selected by a user if these limits are exceeded or subceeded.

9. the software of claim 1 has file transfer protocol which will publish to a secure, password protected intranet or internet website which allows the user to view temperature data of a patient in real-time from any location in the world via a telephone connection, a wireless internet connection, or any method of connecting to the internet.

10. said notification methods of claim 8 can include: an alarm bell; an alarm buzzer; an alarm ringer; an alarm horn; a telephone call; an e-mail; a pager; a fax; or any method of notification system developed in the future.

11. said software of claim 1 allows the user to view the core body temperature and external body temperature of a patient in the operating room during surgery.
